(19) United States
(12) Patent Application Publication
(10) Pub. No.: US 2018/0338712 A1
(43) Pub. Date: Nov. 29, 2018

(54) MUTLI-PROBE MICROSTRUCTURED ARRAYS

(71) Applicant: Imperial Innovations Limited, London (GB)

(72) Inventors: Tony Cass, London (GB); Sanjiv Sharma, London (GB)

(73) Assignee: IMPERIAL INNOVATIONS LIMITED, London (GB)

(21) Appl. No.: 15/326,828

(22) PCT Filed: Jul. 17, 2015
(86) PCT No.: PCT/GB2015/025090

§ 371 Date: Jan. 17, 2017

(30) Foreign Application Priority Data
Jul. 17, 2014 (GB) 1412696.5

(51) Int. Cl. A61B 5/145; A61B 5/1486; A61B 5/00

(52) U.S. Cl. CPC A61B 5/14532; A61B 5/14865; A61B 2562/125; A61B 2562/046; A61B 5/685

(57) ABSTRACT

A microstructured electrode array comprising a base (12) with a plurality of groups of probes (14) formed thereon, each group (13) of probes having a conductive layer formed thereon so that the group (13) forms a single electrode, wherein the electrodes are electrically isolated from each other.

### FIELD OF THE INVENTION

This invention relates to microstructured arrays, in particular electrode arrays, which may be used in sensors, such as biosensor arrays arranged to be placed in contact with the skin to sense analyte(s) in or under the skin, in particular in the dermal interstitial fluid, or may be used in stimulation devices arranged to apply electrical stimulation signals to the tissue or specialized cells such as neural cells.

### BACKGROUND TO THE INVENTION

Continuous monitoring of analytes and metabolites is of great significance in understanding physiological states and in the management of various diseases. An established example is that of continuous glucose monitoring (CGM). CGM has been reported to have a major impact on the management of Type 1 diabetes. However, the three main factors that have limited the widespread adoption of existing continuous glucose monitoring systems (and by implication other continuous monitoring situations) are their lack of accuracy, limited user friendliness and cost. In terms of analytical accuracy, current CGM technologies exhibit a median absolute relative deviation (MARD) between 12.8% to 19.7% and a percentage absolute relative deviation (PARD) of 15-16%. The variability of CGM readings lead to false alarms which diminish the compliance with CGM sensors use. This can be addressed through development of accurate and precise CGM technologies.

### SUMMARY OF THE INVENTION

The present invention provides an electrode array, which may be a micro-structured electrode array, comprising a base with a plurality of groups of probes formed thereon, each group of probes having a conductive layer formed thereon so that the group forms a single electrode, wherein the electrodes are electrically isolated from each other.

The conductive layer may be formed, for example, of platinum, gold, silver, or a carbon based material such as carbon nanotubes or graphite.

The present invention further provides a sensor device, which may be a biosensor device, comprising a base with a plurality of groups of probes formed thereon, each group of probes having a conductive layer formed thereon so that the group forms a single electrode, wherein the electrodes are electrically isolated from each other, and processing means having an input connected to each of the electrodes to receive a signal therefrom, and arranged to process the signals to generate a sensor output, which may be in the form of an output signal.

Each probe may be a single functional device or formation. It may be tapered to a point, for example so that it can pierce the surface of the skin. The tip diameter of the probe may be in the range of 10-50 microns and the height of the microprobe may be in the range of 100 to 1000 microns.

The group of probes may form an array, in which each probe may be an independent entity in respect of its functional properties.

The sensor device may be arranged to convert a physical or chemical parameter to an electrical signal.

The sensor device may be a biosensor, and may be arranged to measure a biological signal or to use biological materials for its function.

One of the electrodes may be arranged to form a reference electrode. At least one of the electrodes is arranged to form a sensing electrode. The at least one sensing electrode may be functionalized, and may be arranged to produce a signal indicative of the presence of an analyte. The reference electrode may not be functionalized, or may be functionalized differently from the sensing electrodes.

The sensing electrode may comprise a probe or array of probes, which makes contact with the analyte. Preferably it should be arranged to apply the desired potential in a controlled way and facilitate the transfer of charge to or from the analyte.

The reference electrode may be a probe or array of probes controlling the sensing electrode's potential and it may be arranged to pass no current at any point.

Two of the electrodes may be functionalized in different ways. The two electrodes may be arranged to detect the presence of different analytes. At least two of the electrodes may be functionalized in the same way. The processing means may be arranged to generate an average of the signals from the at least two electrodes.

At least three of the electrodes may be functionalized in the same way. The processing means may be arranged to identify the signal from at least two of the at least three electrodes as being most similar to each other. The processing means may be arranged to base the output at least predominantly on the signals from those two electrodes.

One or more of the sensing electrodes may be functionalized with alternative enzyme to respond to the presence of a second or multiple other analytes. The processing means may then be arranged to compare the signals so as to generate a sensing output indicative of the presence of the other of the analytes.

At least one of the electrodes may be functionalized so as to act as a compensation electrode to measure the contributions from interfering species present in the matrix. For example in one of the example of continuous glucose monitoring, the presence of electro-active analytes present in the skin compartment to compensate for the interference. As another example one of the working electrodes may be used to measure the oxygen tension in the analyte matrix under examination.

The processing means may be arranged to correct the signal values obtained from sensing electrode, using the signal from the compensation electrode, to nullify the interference effect or to take into account the effect of oxygen concentration in the biological matrix.

The compensation electrode may therefore be an electrode that corrects for the other interferants present in the matrix under investigation.

One of the electrodes may be arranged to form a counter electrode, which may be arranged to pass all the current needed to balance the current observed, or flowing, at the sensing electrode.

The processing means may be arranged to analyse the signals and detect the occurrence of a predetermined change in the signals over time.

The sensor/biosensor may comprise several electrodes that are monolithically integrated on the same structure. The sensor may be arranged to enable continuous monitoring of glucose and/or other analytes such as lactate, for example in the dermal interstitial fluid, and preferably in a minimally invasive and pain free manner. The array may be sub-divided into a reference electrode/counter electrode, a background correction electrode and other sensing electrodes that are functionalized to measure glucose and other analytes simultaneously. The multielectrode arrays add redundancy to the platform thus enabling voting methods (either hardware or software) to improve the accuracy and precision of the device.

The functionalization may comprise electro-polymerisation of phenol, for example on the platinum microprobe array electrodes, optionally with glucose oxidase entrapped in the electro-polymerised polyphenol. Alternatively the functionalization, for example of a gold electrode, may comprise forming a self-assembled monolayer of thiols, optionally followed by immobilisation of glucose oxidase enzyme. A membrane may be deposited on the microprobe array electrodes.

The invention further provides a stimulation device comprising an electrode array according to the invention, a power source, and control means arranged to generate a stimulation signal from the power source and control the application of the stimulating signal to each of the electrodes.

The invention further provides a method of producing a probe according to the invention. The method may comprise making a master, for example a metal master, e.g. of aluminium, making a mould, for example a PDMS mould from the master, and then forming a sensor array, for example from epoxy, in the mould. The electrodes may then be formed on the sensors by forming a mask on the sensor body that defines a number of openings corresponding to the areas to be covered by the electrodes, and then applying the conductive coating to the areas exposed by the mask to form the electrodes. The processor may then mounted on the back of the sensor body and electrically connected to the electrodes.

The device may further comprise, in any combination, any one or more features of the preferred embodiments of the invention, which will now be described by way of example only with reference to the accompanying drawings.

### BRIEF DESCRIPTION OF THE DRAWINGS

FIG. 1 is a sectional view of a biosensor device according to an embodiment of the invention;

FIG. 2 is a front view of the biosensor device of FIG. 1;

FIG. 3 is a SEM image of part of the device of FIG. 1;

FIG. 4 is a set of cyclic voltammograms obtained from the three bare working electrodes against an integrated Ag/AgCl reference electrode with FCA as the redox mediator; and

FIG. 5 is a set of dose-response curves obtained after functionalization of a single working electrode.

### DESCRIPTION OF THE PREFERRED EMBODIMENTS

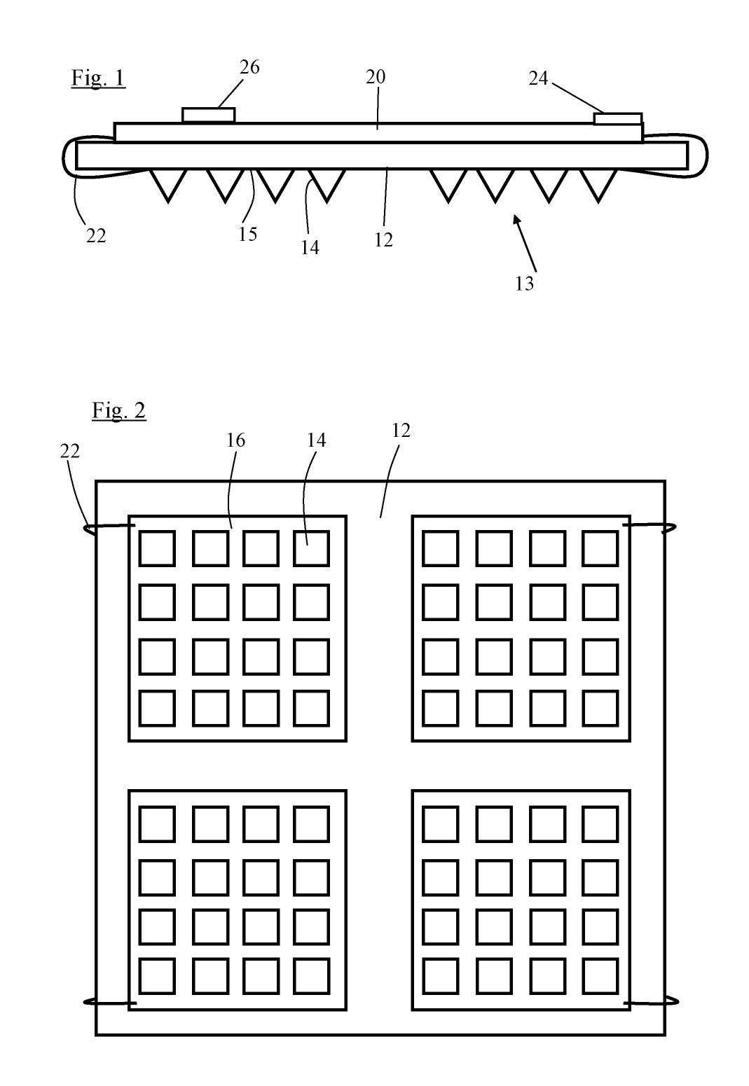

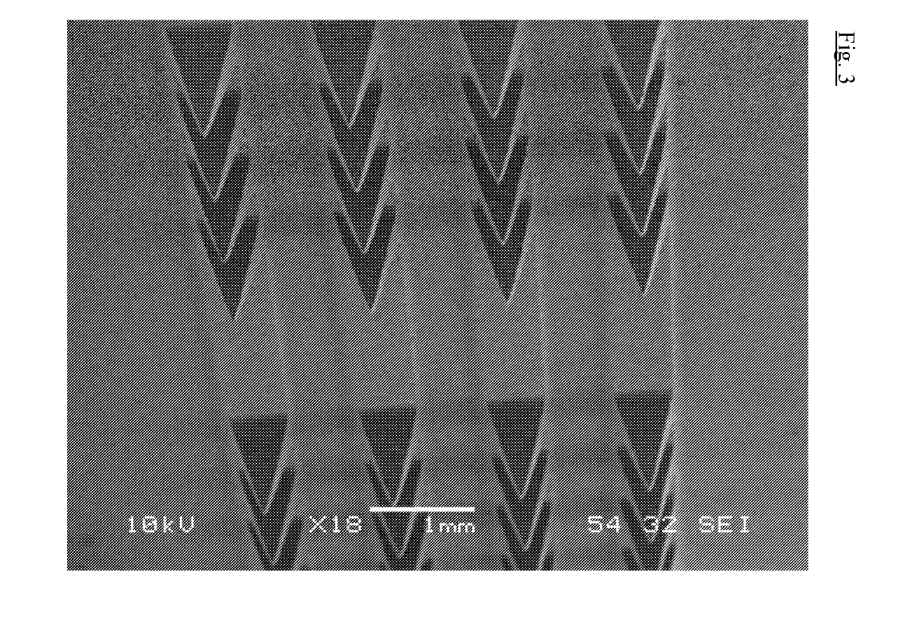

Referring to FIGS. 1 and 2, a biosensor device according to an embodiment of the invention comprises a main sensor body 10 made up of a square base 12 with four groups 13 of microstructures, which in this case take the form of microprobes 14 formed on its front surface 15. The base 12 and microstructures are formed as a single epoxy moulding. Each of the groups of microstructures 14 is arranged in a 4×4 square pattern. Each of the microstructures is in the form of a four-sided pyramid whose height is approximately equal to the width of its base, both of which are very approximately 0.5 mm in this embodiment, though of course the exact size and shape will vary depending on the application of the sensors.

Each group 13 of microstructures 14, together with the area of the base 12 between the microstructures in the group, is coated with a conductive coating so that together they form a single electrode 16. The area of the base 12 between the groups is not so coated, and the four groups 13 therefore form four electrodes 16 which are all electrically isolated from each other. Three of the electrodes 16 are also functionalized to make them responsive to the presence of a particular analyte, which generally includes applying a substance to the surface of the microstructures 14 that will react with the analyte to produce a detectable signal on the electrode. One of the electrodes is not so functionalized and forms a reference electrode.

A processor 20 is mounted on the back of the base 12, i.e. on the opposite side to the microprobes 14, and each of the functionalized electrodes and reference electrode 16 is connected to a respective input of the processor by a respective conductor 22. Although shown schematically, the conductors 22 are most easily formed on the surface of the base 12. The processor 20 is arranged to detect the signals of the functionalized electrodes, and process them to generate an output at an output 24 of the processor.

Fabrication of the device can be carried out in a variety of ways such as injection moulding, hot embossing, laser micromachining, 3D printing and micromoulding.

In some embodiments, the process comprises making moulds, for example PDMS moulds, from metal masters, for example aluminium masters, and then forming epoxy microstructured sensor arrays in the moulds. The electrodes are then formed on the sensors by forming a mask on the sensor body that defines a number of openings corresponding to the areas to be covered by the electrodes, and then applying the conductive coating to the areas exposed by the mask to form the electrodes. The processor is then mounted on the back of the sensor body and electrically connected to the electrodes. The steps used in one particular embodiment are set out below.

Preparation of Aluminium Masters

The aluminium master is produced by electric discharge milling of aluminium blocks. The master corresponds in shape to the final probe which is described in more detail below.

Preparation of PDMS Moulds of the Microprobe Array Structures

1. Remove the aluminium masters from a sealed petri dish. Inspect the aluminium masters under a microscope to ensure there are no remains of any PDMS elastomer, clean thoroughly with deionised water and dry with nitrogen.

2. Clean thoroughly the black epoxy moulds with Deionised water and dry with blue roll (paper towel) and nitrogen. Place the aluminium masters in the epoxy moulds using a double-sided adhesive tape.

3. Prepare the elastomer solution using the commercially Sylguard 184 kit consisting of the elastomer and the curing agent. Mix the elastomer and the curing agent in the ratio of 10:1 (w/w). Weight 50 grams of the elastomer in a disposable shallow plastic dish, for example a Petri dish, and add 5 grams of the curing agent. This weight of materials allows the manufacture of four PDMs moulds.

4. Store the PDMS mix in a freezer at −20 C for 15 minutes.

5. In a clean fume hood, pour the cold solution on to the mould holding the masters and allow it to set over a period of 48 hours.

6. Once the elastomer sets, using a fresh, sterile surgical blade cut and peel the PDMs mould. Repeat steps 1-4 till eight PDMS moulds are manufactured.

7. Store the PDMS moulds in a clean container sealed with parafilm, before use to avoid any contamination or exposure to dust particles.

Manufacture of SU 8 50 Epoxy Microprobe Structures from the PDMS Elastomer Moulds

1. Several microprobe array structures can be manufactured in each batch. Each batch takes approximately 3 hours to manufacture.

2. On a clean bench, open the eight sealed PDMS moulds and using a plastic dropper introduce the SU 8 50 epoxy to the moulds. Place the petri dish (without the lid) in a clean desiccator, seal it and apply a vacuum for 5 minutes to remove air bubbles.

3. Remove the samples from the desiccator, cover the dish with a lid and load it on to the swing bucket rotors designed for the Eppendorf centrifuge 5810/5810Rs. Each bucket rotor can hold two petri dishes with the moulds. Use a Teflon tape to hold them in place.

4. Clean the centrifuge with 70% Ethanol solution before use. Set it at 4° C. and leave it till it reaches 4° C. Load the buckets in the centrifuge and spin at 200 rpm for 1 minute. Next, spin at 4000 rpm for 30 minutes.

5. On completion, remove the petri dishes from the centrifuge and put it in a clean UV chamber with an ultraviolet transilluminator. Ensure that chamber and the transilluminator are cleaned before and after use. Set the wavelength at 365 nm and leave the petri dish in the chamber for two hours without the lid, for the epoxy to crosslink.

6. After two hours inspect the completion of crosslinking. Feeling the base of the structures first does this; it should be hard and peel off easily from the PDMS mould.

7. If it does not feel hard enough and does not peel then leave it in the chamber for another 60 minutes.

8. Peel off the epoxy microprobe arrays from the PDMS.

9. A full inspection of the microprobe array structures (one from each batch) should be done by Scanning electron microscopy.

10. Transfer the microprobe arrays to a fresh, sterile 6 well tissue culture plate. Label the top of the plate using a permanent marker pen and seal off the 6 well plate using parafilm till further use.

Metallisation of the Microprobe Array Structures:

We give here two examples of metallisation. The first is for producing a microprobe electrode array with platinum working and counter electrodes and a Ag/AgCl reference electrode. The second is for producing an array with gold working and counter electrodes and a Ag/AgCl reference electrode. Many of the steps are common to both methods.

1. Transfer the six well plates to facility for metallisation.

2. Mask the electrode arrays with a masking tape or parafilm to selectively deposit the metals in the required locations.

3. Stick the samples on a fresh silicon wafer using a double sided tape and load the silicon wafer onto the sample holder. Blow with nitrogen to ensure no dust particle or debris is carried into the metallisation chamber for example the BOC Edwards electron gun evaporation and sputtering system or a similar system.

4. The chamber in which the device is metallised is controlled and subjected to inert gases such as nitrogen and argon. The metallisation is done at low pressure (2×10⁻⁶ Bar). First mask the bare microprobe array electrodes that will function as working electrodes or counter electrodes. Set conditions to sputter 150 nm of silver.

5. Once the silver microprobe array electrode is ready mask it with parafilm and leave the microprobe array electrodes, that will function as working electrodes, exposed.

6. Set conditions to get a 15 nm titanium layer followed by a 50-100 nm layer of platinum to obtain Platinum microprobe array electrodes.

7. For gold microprobe arrays set conditions to get a 15 nm adhesion layer and 100 nm of gold.

8. After metallisation to form the working electrodes, the silver microprobe array electrode can be modified to a silver/silver chloride reference electrode by treating the electrode with a saturated solution of Ferric chloride or by electrochemical modification.

Bonding Wire of the Metallised Microprobe Array Structures and Encapsulation

1. Cut wires from wire rolls (internal diameter 0.3 mm, ROHS compliant), strip the outer plastic covering to expose the metallic bits at the tips. Bond the wires to the devices using a silver loaded electrical conductive paint (RS components). Allow it to dry for 15 minutes.

2. Once dry cover the connection using a fast setting epoxy adhesive (Araldite) and let it set over 30 minutes.

3. As examples of encapsulation of gold microprobe array electrodes, spin photoresists such as the AZ photoresist or the SU 8 epoxy material.

Functionalization of the Metallised Microprobe Arrays

Functionalization of the metallised microprobe arrays can be done in many different ways depending on what the sensor is to be used for. We describe here two different methods for functionalization of the microprobe array electrodes, each of which can be used to make them suitable for use as amperometric glucose biosensors. The first method involves electropolymerisation of phenol on the platinum microprobe array electrodes with glucose oxidase entrapped in the electropolymerised polyphenol. The second method is for functionalization of gold microprobe array electrodes using self-assembled monolayer of thiols followed by immobilisation of glucose oxidase enzyme and depositing a membrane conformally on the microprobe array electrodes. The membrane can also be deposited on the electrodes functionalized by the first method.

Electropolymerisation of Platinum Microprobe Array Electrodes

1. Deposit small volumes (200-3004) of 50 mM phenol solution containing 10 mg/mL of the enzyme, for example Glucose oxidase in case of glucose biosensor device on the metallised devices.

2. The platinum working electrodes should be poised at 0V for 20 seconds and then biased at 0.9V for 15 minutes using a potentiostat, this cycle can then be repeated six times.

3. The performance of the resulting sensors can then be evaluated using chronoamperometry, whereby 2004 of the analyte solution is pipetted onto the array and, following the application of a potential of 0.7V (against the integrated Ag/AgCl reference electrode), the current is recorded for 60 seconds. The current (at t=60 s) vs. concentration can then be plotted.

Gold Nicroprobe Array Electrodes Based Glucose Biosensor Devices

1. Prepare a 40 mM solution of Thiomalic acid by dissolving 0.34 grams of Thiomalic acid in 50 mL of deionised water. Place the solution in a clean glass container and dip the microprobe arrays in the thiomalic acid solution for 2 hours.

2. Prepare 2 mM 1-Ethyl-3-(3-dimethylaminopropyl) carbodiimide (EDC) solution by weighing 0.03 grams. For the 5 mM N-hydroxysuccinimide (NHS) weigh 0.02 grams. Add it to 50 ml of deionised water in a sterile eppendorf tube. The thiomalic acid layer is activated to couple with the enzyme Glucose oxidase using EDC and NHS. Immerse the microprobe arrays with SAM layer in 50 ml of the EDC-NHS solution in a beaker for 2 hours, in a fume hood.

3. Prepare 40 ml of 10 mg/ml solution by weighing 400 mg of Glucose oxidase and dissolve it in 0.1 M PBS solution. Incubate the microprobe arrays with glucose oxidase solution (10 mg/mL in 0.1M PBS) overnight at 4° C.

4. Leave the microprobes for drying for 1 hour in a clean fume hood.

5. Prepare the epoxy polyurethane by dissolving 17.8 mg each of Part A and Part B of ATACS5104 epoxy adhesive and 26.7 mg of PU in 4 ml of THF and 1 ml of Brij 30. The membrane layer is conformally deposited by dipping the microprobe arrays in the epoxy polyurethane solution for 15 seconds. (This membrane can be deposited on the electrodes functionalized with polyphenol also.)

6. Leave the microprobe array devices for drying for 30 minutes in a cold room before packing them into Sterile 50 ml Eppendorf tubes.

7. Store the devices in 4° C. cold room before further packaging and sterilisation.

In Vitro Studies

Cyclic voltammetry (CV) showing multielectrode system showing integrated reference electrode: The monolithically integrated microprobe arrays were tested by performing cyclic voltammetry using ferrocene carboxylic acid (FCA) as a redox species. The metallised microprobe arrays comprised four 4×4 sub-arrays. One of these sub-arrays was metallized with silver, and the other four were conformally sputtered with 150 nm gold.

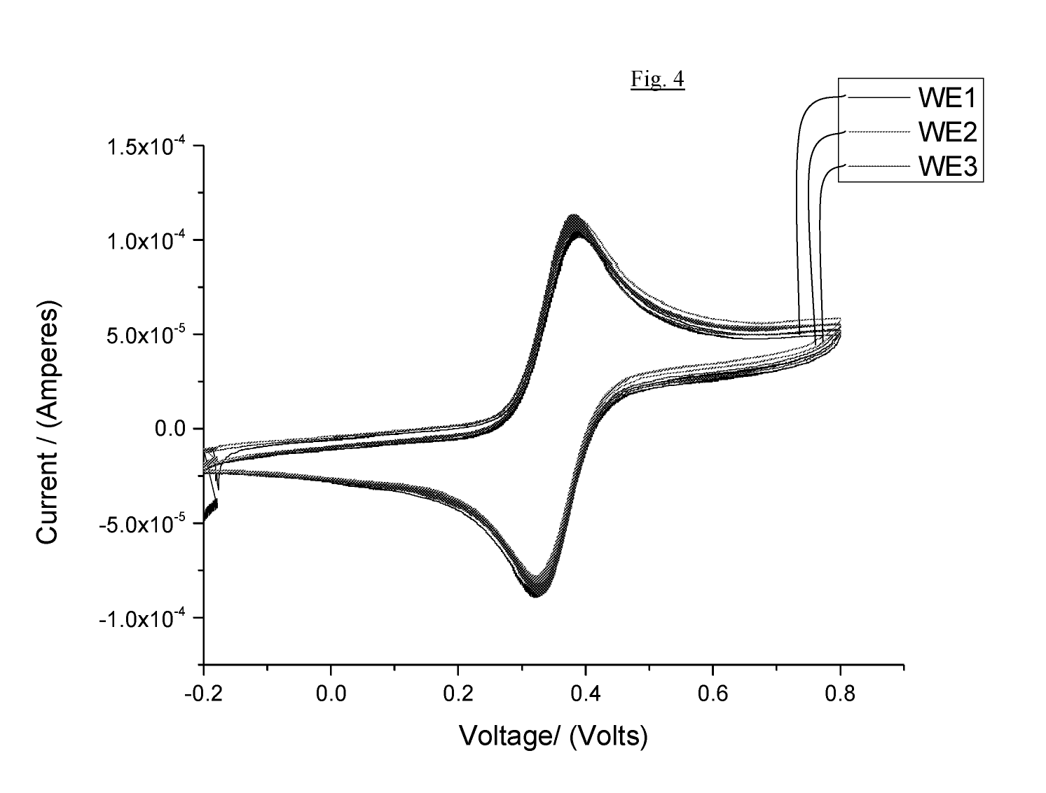

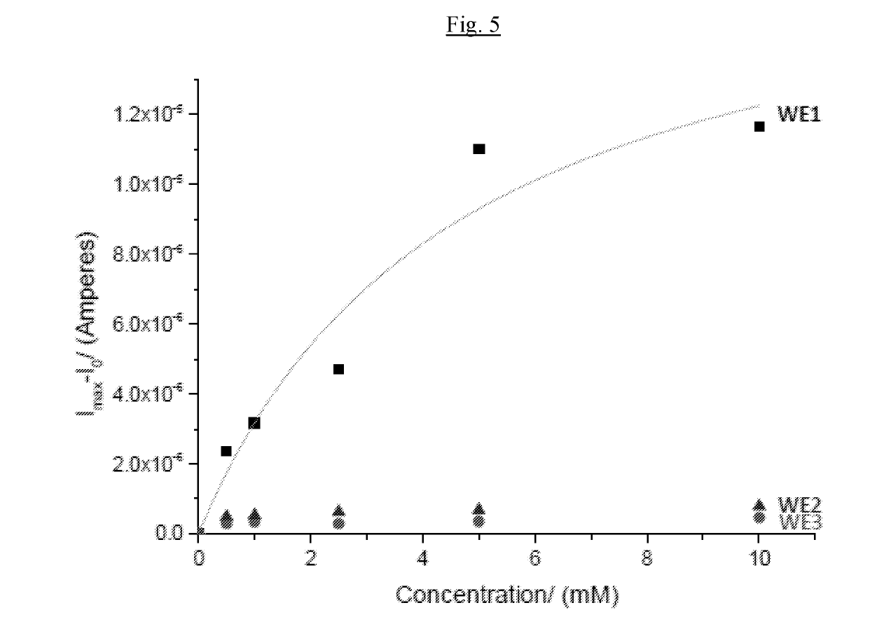

The silver microprobe sub-array was modified to Ag/AgCl reference electrode using a saturated Ferric chloride solution and found to be very stable. FIG. 4 is a set of cyclic voltammograms obtained from the three bare working electrodes against the integrated Ag/AgCl reference electrode with FCA as the redox mediator. As shown in FIG. 4, the bare electrodes exhibit quasi-reversible electrochemistry (ΔEₚ=100 mV at 100 mVs⁻¹ scan rate) and reproducibility (2.5% variation in Iₚ) FIG. 5 is a set of dose-response curves obtained after functionalization of a single working electrode (WE1-squares) with glucose oxidase. WE2 (triangles) and WE3 (circles) are blank electrodes. (Michelis-Menten constant K_{M}=12.25 mM, maximum limiting current Iₘₐₓ=15 μA). As can be seen, the functionalization of one electrode does not generate any response in the other electrodes, and this lack of chemical cross-talk between the electrodes shows that they can be functionalized in different ways and readings from each electrode will not be affected by the others.

The processing performed by the processor 20 will depend on the manner in which the electrodes have been functionalized. In this embodiment there are three working electrodes, which have been functionalized in the same way, and one reference electrode. The processor is arranged to record sample values of each of the electrode signals taken at a regular sample frequency. The processor 20 is then arranged for each sample time to determine for each of the working electrodes a normalised signal value by determining the difference between the working electrode and the reference electrode. The processor is then arranged to compare the three normalised values and determine an average value for all three and record that as an average signal for that sample time. In a modification to this arrangement, prior to calculating the average, the processor can be arranged to determine a measure of dissimilarity of each of the signals of the three working electrodes from the other two, and if the degree of dissimilarity for one of the signals exceeds a predetermined level, to discard that signal in calculating the average output value.

Once the sequence of sample values is recorded, the processor is arranged to output the average value at the output 24 as a real time output. However it is also arranged to analyse them to determine whether the variation on the average signal over time meets any of a number of predetermined conditions. These may include, exceeding a predetermined rate of change with respect to time, exceeding a predetermined threshold, or falling below a predetermined threshold. If one of these conditions is met, then the processor is arranged to transmit a warning signal via the Bluetooth transmitter 26.

It will be appreciated that the analysis of the electrode signals can be carried out in a variety of ways depending on the type of test that is being done. The averages can be calculated in different ways or over different time periods, either the real time signal output, or the analysis of stored sample values can be omitted.

In a further embodiment, one of the three sensing electrodes functionalized in a different way from the other two so as to act as a compensation electrode. For example if the probe is to be used to monitor glucose, the two sensing electrodes can be functionalized to respond to glucose, but will typically also respond to other analytes or ‘interfering species’. Therefore the compensation electrode functionalized so as to respond to the interfering species, but not to glucose. In this case the controller is arranged to compare the signals from the compensation electrode with those from the sensing electrodes to generate two compensated sensing signals, which are then averaged to generate an output. The signal from the reference electrode is used to normalize the signals from each of the three other electrodes prior to the comparison. The output value is then generated at the output 24 of the device.

In a further embodiment, a stimulation electrode device comprises the electrode array structure of FIGS. 1 and 2, but without functionalization of the electrodes 16. The processor 20 is connected to a power source and arranged to generate a stimulating signal from the power source and to apply the stimulating signal to the electrodes 16 so as to provide stimulating signals at the electrodes. The provision of separate electrodes on the same device allows different signals to be applied to each of the electrodes and therefore to different areas of the skin. In a modification to this embodiment, the processor is not present on the device, and the electrodes are connected to a remote power supply and controller, which applies the stimulating signals to the electrodes.

1. A microstructured electrode array comprising a base with a plurality of groups of probes formed thereon, each group of probes having a conductive layer formed thereon so that the group forms a single electrode, wherein the electrodes are electrically isolated from each other.

2. A sensor comprising a microstructured electrode array according to claim 1, and a processor having an input connected to each of the electrodes to receive a signal therefrom, and arranged to process the signals to generate a sensor output signal.

3. A sensor according to claim 2 wherein one of the electrodes is arranged to form a reference electrode and counter electrode and at least one of the electrodes is arranged to form a sensing electrode.

4. A sensor according to claim 3 wherein the at least one sensing electrode is functionalized so as to produce a voltage/current signal indicative of the presence of an analyte, and the reference electrode is not so functionalized.

5. A sensor according to claim 2 wherein two of the sensing electrodes are functionalized in different ways to respond to the presence of different analytes.

6. A sensor according to claim 2 wherein at least two of the sensing electrodes are functionalized in the same way and the processor is arranged to generate an average of the signals from at least two electrodes.

7. A sensor according to claim 2 wherein at least three of the sensing electrodes are functionalized in the same way and the processor is arranged to identify the signal from at least two of the at least three electrodes as being most similar to each other, and to base the output signal at least predominantly on the signals from those two electrodes.

8. A sensor according to claim 5 wherein one of the sensing electrodes is functionalized to respond to the presence of at least two analytes, and one of the electrodes is a compensation electrode functionalized so as to respond to only one of the two analytes, and the processor is arranged to compare the signals so as to generate a sensing signal indicative of the presence of the other of the analytes.

9. A sensor according to claim 2 wherein the sensing electrodes are usable to diagnose the health or functioning of the sensor.

10. A sensor according to claim 9 wherein impedance measurements are used to check whether the sensor is performing optimally.

11. A sensor according to claim 2 wherein the processor is arranged to store a sequence of sample values of the signals and to analyse the sample values.

12. A sensor according to claim 2 wherein the processor is arranged to analyse the signals and detect the occurrence of a predetermined change in the signals over time.

13. A stimulation device comprising an electrode array according to claim 1, a power source, and a controller arranged to generate a stimulation signal from the power source and control the application of the stimulating signal to each of the electrodes.

14.-16. (canceled)
